(19) United States
(12) Patent Application Publication
(10) Pub. No.: US 2013/0197216 A1
(43) Pub. Date: Aug. 1, 2013

(54) Device And Method For Extracting Active Principles From Natural Sources, Using A Counter-Flow Extractor Assited By A Sound Transduction System

(71) Applicants: María Graciela Cares Pacheco, Central Santiago (CL); Luis Francisco Javier Gaete Garreton, Central Santiago (CL); Yolanda del Pilar Vargas Hernández, Central Santiago (CL); John Gabriel Alarcon Camacho, Camino Internacional Con Con (CL); Javier Ignacio Sainz Lobo, Camino Internacional Con Con (CL)

(72) Inventors: María Graciela Cares Pacheco, Central Santiago (CL); Luis Francisco Javier Gaete Garreton, Central Santiago (CL); Yolanda del Pilar Vargas Hernández, Central Santiago (CL); John Gabriel Alarcon Camacho, Camino Internacional Con Con (CL); Javier Ignacio Sainz Lobo, Camino Internacional Con Con (CL)

(73) Assignee: NATURAL RESPONSES S.A., Región de Valparaiso (CL)

(21) Appl. No.: 13/520,732

(22) PCT Filed: Jan. 3, 2011
(86) PCT No.: PCT/CL2011/000001

§ 371 Date: Dec. 17, 2012

(30) Foreign Application Priority Data
Jan. 4, 2010 (CL) 03-2010

(51) Int. Cl. B01D 11/02; C07H 1/08

(52) U.S. Cl. CPC B01D 11/0261; C07H 1/08
USPC 536/128; 422/127; 422/128

(57) ABSTRACT

The invention relates to a device and method for extracting active principles from natural sources, using a counter-flow extractor assisted by a sound transduction system, which allows a cavitation sound field to be applied in the zone containing the material formed by the raw material of the natural product and a solvent extraction medium. According to the invention, the device (11) comprises: an inclined casing (12) containing a helical screw conveyor (13) having a plurality of blades (17), said casing including a lower end (15) and an upper end (16); a hopper (21) for introducing the material, which is disposed on top of the lower end (15) such that it is inclined at an angle # in relation to the surface of the casing (12), said feed hopper (21) including a second helical screw conveyor (22); an outlet hopper (31) for releasing the treated material, which hopper is located at the upper end (16) of the casing (12); a first load line (23) for loading the solvent extraction medium, which load line is located at the upper end (16); a discharge line (26) for discharging the liquid extract, which discharge line is located at the lower end (15) and is provided with a screen (27) that filters the liquid extract; and a sound transduction system (29) for producing ultrasound, which is located at the lower end (15) on a surface portion (28). The method comprises the following steps: (a) preparing the raw material; (b) supplying the product and the solvent to the device, in counter-flow mode; (c) applying a sound field to the product together with the solvent; (d) extracting the product with the extraction liquid, in counter-flow mode; and (e) collecting the liquid extract and the depleted material.

### BACKGROUND OF THE INVENTION

The present invention relates to a method for extracting active principles from natural resources.

When extracting active principals from natural sources, many times is preferable to extract them at low temperatures, low liquid-solid ratio and low process time. The idea is to prevent the degradation of the components and subsequent excessive concentrations (relative). However, these simple exposed restrictions cause devastating effects on the performances of processes developed nowadays. Sub-optimal solutions of processes are frequently used which privilege performance over quality, even without achieving optimal performances and producing degradation of the components due to the use of high thermal power.

Currently, obtaining active principals from natural sources is made through the use of techniques type batch or through continuous extractions. The extraction alternatives currently available in the market and references to the present invention are as follows:
1. Batch Systems of countercurrent extraction.
2. Continuous Extraction Systems.
3. Ultrasonic Extraction.

The most used systems in the industry are extractions type batch, these processes have multiple applications related to extraction stages in tanks with re-circulation that can be adapted to simulate a semi-continuous countercurrent process. However, batch extractor do not take full advantage of the mass gradients among the components, they use high liquid-solid ratios and multiple washes using this way, great amount of solvent per unit of extracted solid.

Currently, continuous extraction systems work mainly in the form of “countercurrent”, this means that the component that delivers (solute, raw material, etc.) is with the solvent that collects, in opposite direction inside the extractor. Thus, an infinitesimal amount of substance to be extracted is always with an amount of solvent without saturating, avoiding the achievement of balance during the process to be suspended.

The continuous systems with screw have multiple applications and there are several promotions of equipments offered to companies Worldwide. This type of processes is commonly used in the food industry to separate soluble fractions of several types of raw material because they offer a minimum use of dissolvents and the capacity of recovering the extract in high concentrations. There is a considerable amount of documents in which this type of extractors and/or their methods of use are described, the most relevant for this invention is US1995/5409541 “Method and apparatus for extracting soluble and dispersible materials from products using a slotted scroll extractor” of David R. Walter. In the license, it is disclosed a method and apparatus for the extraction of soluble and insoluble of products through a Conveyor screw extractor, more precisely, the apparatus is comprised of a helical screw contained within a case formed by a hallow half cylinder made of stainless steel and equipped with semicircular walls at the ends to retain the material under treatment. The case is surrounded by a system which, forming a double wall along the reactor, allows the temperature control of it. Additionally, the apparatus has the capacity of incorporating different solvents in different sections of the screw, obtaining a process with the characteristic in a first stage where the solvent is water and in a second stage it is alcohol. In summary, the invention disclosed in the document US1995/5409541 shows a simple method of extraction and an apparatus to carry it out, which allows a multi-stage extraction where different means of extraction can be used and/or different inflow caudal of the solvent in the different sections of the extractor.

The countercurrent apparatus have shown positive results, however, the process does not meet the optimal standards. The source material tends to agglomerate in the case of the equipment forming a compact layer, and the solvent tends to flow along the surface of it without facing the efficient form of the raw material, leaving in it, a significant part of the active principals sought. Another of the most significant disadvantages is the high amount of solvent needed to improve the process, since the diffusion only takes place optimally in those screw sections where the source material is partially submerged. To solve these problems, sub-optimal solutions of process are used, for example: the possibility of re-enter the source material (or extract) to the equipment in order to obtain improvements in the efficacy of the process. However, there are some natural sources that are affected by the growing of microorganisms that produce toxins which in some applications are not allowed. To eliminate the toxins these microorganisms produce in the final extract, a more delicate and technically more complicated process must be performed, which is used to be performed in costly processes (high temperature, membranes). There are other continuous extraction systems with application of microwaves. The procedures of extraction of the natural products by microwaves described in the literature can be implemented only on small biological masses due to the applied microwaves power, between 10 to 20 [kW] per kilogram of biological material using water as a solvent. In consequence, these procedures cannot be implemented on an industrial scale, because they make the use of the technique prohibitive due to requirements of investment and power.

In WO/1994/026853 “Method and plant for solvent-free microwave extraction of natural products” of Philippe Mengal and Bernard Mompon it is disclosed a method and apparatus for the extraction of natural products from biological material. The invention consist of subjecting the biological material, in absence of solvent, to radiation through microwaves (2450 [MHz]). This allows the hydro distillation of the material to be extracted, not because of the water brought from outsider Turing the process, but for the water contained in the biological material treated (around a 30% in biological materials). The invention described in the document WO/1994/026853 has comparative advantages as per the traditional methods of extraction by microwaves; it is obtained a sample free of all dissolvent residues, high performances compared with the traditional process of hydro distillation. Apart from having a low cost of application, this technique has the advantage of allowing the acquisition of products extracted free of residual dissolvents and, consequently, it does not need further treatments to eliminate such residues. It is important to mention that there are several products that cannot be extracted from the natural material that contains them, according to such methods, and for those that need extraction techniques using organic dissolvents.

The use of ultrasounds to assist the extraction of bioactive principals from plants has been considered in several studies, there are studies about the application of ultrasounds for extraction since the 50s, decade where the use of ultrasounds in diverse processes started to be strongly explored. Contemporary studies such as the one of Mircea Vinatoru described in the article “An overview of the ultrasonically assisted extraction of bioactive principles from herbs”, Ultrasonics Sonochemistry, 2001, vol. 8, 303-313, in which it is presented a complete comparison of the different methods of extraction of vegetal material developed until now (Distillation, Extraction by solvents, Cold compression and non conventional techniques) where the capacity of the ultrasounds are highlighted when quantifying efficiency in the processes. Additionally, in his work, Vinatoru discloses the firs industrial reactor for extracting vegetal products by ultrasonic assisted. The investigation was held by the Programme EU COPERNICUS (ERB-CIPA-CT94-0227-1995). The apparatus has 1 m³ capacity, has 4 transducers that radiate from the reactor's walls. The invention disclosed by Vinatoru gave efficiency improvements in the process in a 50% compared to the classical process.

Currently, it is accepted that ultrasounds can stimulate diverse processes of extraction, in the work of Kamaljit Vilkhu specially his article “Applications and opportunities for ultrasound assisted extraction in the food industry, a review”, Elsevier, 2008, vol 9 161-169, it is explained that the implosion of the micro bubbles generates macro turbulence, collision between particles and disturbance in the micro pores of biomass particles which accelerate the internal diffusion and “Eddy” diffusion. The improvement in the efficiency of the processes of extraction assisted by ultrasound is attributed to the propagation of high-intensity ultrasonic pressure waves in the fluids particularly the phenomenon of acoustic cavitation. It is important to mention that in order to increase the efficiency of the process assisted by ultrasounds, the size of the material in extraction must be reduced, due to there is a relation between the size of it and its interaction with the acoustic field defined in the reactor, as it is shown in the article of Balachandran, Kentish, Mason and Ashokkumar (2006), “Ultrasonic enhancement of the supercritical extraction from ginger”, Ultrasonics Sonochemistry, vol. 13, 471-479; by reducing the size of the chips, the surface of contact directly exposed to ultrasonic radiation increases, favoring this way, the transference of mass of the soluble components from the source material towards the solvent.

FIG. 1 shows a schematic drawing of a vegetal cell structure (1) in which the material particles (2) migrate to a dissolvent environment (3). Vegetal cells are (4) are separated from the intercellular environment (5) and from the other cells by the cell wall (6). Vegetal cells are delimited by the wall cell (6) and the plasmatic membrane (7). The plasmatic membrane (7) is an structure that surround the cell, define its limits and contributes to keep the balance between the interior and exterior of these, and the cell wall (6) is the layer that goes outside the membrane, avoid shape and position changes, its function is to project the cell. When applying ultrasounds to the extractions, the contact of the solvent with the solid improves because of the cavitation phenomenon of the acoustic waves. These cavitation micro bubbles open spaces (capillaries) and favor the entry of the solvent improving the hydration. Additionally, they allow the extraction of heat-sensitive components due to the decreasing of thermal power and offer a decrease in the duration process. FIG. 2 shows the same vegetal cell structure bombarded by acoustic pressure waves (8); these waves are the ones that generate the acoustic cavitation bubbles. As the cavitation bubbles implode near the solid-liquid inter phase, the fluid jet produced by the implosion, hit the surface of the cell generating macro turbulence, collision between particles and disturbance in the micro pores of biomass particles, causing damage to the cell wall (6) and its subsequent rupture (9) improving, thus, the diffusion (8) and consequently, the extractive process.

In WO/2005/087338 “Process for extraction of diterpenes and triterpenes from biomaterial” of Stevanovic and Lavoie, it is disclosed an ultrasonic process to extract diterpenes and triterpenes from biological material. The invention described in the document, present a comparison between the potentials of two traditional methods of extraction (Soxhlet and Maceration) and the Ultrasonic extraction. The extraction techniques developed use organic dissolvents such as Methanol, Dichloromethane, Hexane and Ethyl Acetate.

The extraction method of Soxhlet causes modifications in certain molecules thermally unstable due to the use of high temperatures. On the other hand, the Maceration techniques studied do not use high temperatures but present less performance in the efficiency of the process. In the experimental cases studied, the ultrasonic extraction presents an increase in the process efficiency obtaining a reduction in the extraction time from 6 days to 30 minutes. However, it is important to mention that the ultrasonic extraction process studied in the document WO/1994/026853 has been established at a laboratory level, for reduced volumes of solution with a radiant face of the transducer of 0.5 [inch]. The implementation of this technique at an industry level due to high acoustic intensities necessary to obtain an efficient energy transmission to high volumes of product would prohibitive the use of this technique.

There are initiatives of ultrasound applications at industrial level, where Batch alternatives have been developed with agitation systems. The industrial application of these processes depends fundamentally on the technology of generation of high-intensity ultrasounds. The main points to consider in the ultrasonic transducers are the power capacity, the performance, the extent and distribution of the vibration and the directionality of the radiation emitted. Economically, it is profitable the introduction of an ad-hoc device in the industrial process and for this, it is necessary the development of specific radiators, this is because probably, in this case, the ultrasonic field is more coherent, the extensions are larger and it is expectable to have more pure field effects than the case of ultrasonic baths, which radiation is substantially diffuse.

Even when the extraction apparatus assisted by ultrasounds have prove positive results, they present disadvantages such as: the active region of the sonication process is restricted to the surroundings of the radiant face of the transducer; the acoustic radiation area must have a high liquid-solid ratio, which reduces the economy in the extraction process due to the need to concentrate later; the generation of ultrasonic power in fluids present problems due to the low acoustic impedance and high absorption of these means. Therefore, to obtain a sufficient energy transmission and produce high intensities, it is necessary to obtain good impedance adaptation between the transducer and the fluid, great extents of the transducer vibrations and a high energy concentration. Due to the high intensities needed to obtain an efficient energy transmission to the process, it is difficult to correct that the volumes of the product be high and the processes be continuous.

For the same reasons previously mentioned in EP/0243220 “Procédé et dispositif d′extraction par ultrasons de produits oléagineaux à partir de graines aléagineuses” of Bernhard René Guillot, it is disclosed an extraction process assisted by ultrasounds. The process consist of diverse extraction sub-processes: (1) Mechanical Treatment of the material (milling, flattening), (2) Percolation or Extraction by dissolvents (system type batch with agitation), (3) Ultrasonic Extraction, (4) Washing stages (at least one) and finally a (5) Micellar treatment to recover the solvent. The described method uses ultrasounds to break the links of the components unextractable from the residual oil by traditional methods (percolation, dissolvents). The extraction process described in the document EP/0243220 obtains practically total extractions of the oily material (99%). However, it is important to mention that this technique does not take full advantage of the mass gradient among the components studied. In the process, multiple extraction and washing stages are performed using great amount of solvent per unit of extracted solid.

This antecedents of the previous art, reveal that the methods and apparatus of countercurrent ultrasonic extraction for obtaining active principles from natural sources are efficient under specific conditions, separately both have disadvantages and restrictions when used at industrial level, either because of high energy costs, amount of raw material to be treated, amount of solvent, type of solvents, high temperatures in the process, long extraction times, etc.

### BRIEF SUMMARY OF THE INVENTION

The present invention refers to a method and apparatus for extracting active principles from natural sources. The key of the present invention consist of the optimal combination of a countercurrent extraction system by a screw with the application of an acoustic field. The invent refers to a continuous extraction in a device in which through one end the source material goes in as the fluid is inserted through the other end on countercurrent, allowing along the continuous extractor to vary the extraction parameters, for example, temperature, solid-liquid relation and residence time. The use of ultrasounds improve the extraction performances at a given temperature allowing the use of low liquid-solid relation, which makes the extractor to have better performances in relation to a regular continuous extractor.

The present invention has comparative advantages significantly superior to methods and apparatus currently used, its power consumption is lower, using less solvents, by working at a lower temperature reduces the need of thermal power, uses a low solid-liquid ratio, minimize the area and necessary section for the application of acoustic power, it requires less applied acoustic power decreasing the magnitude of investment required for its installation and operation expense.

Continuous extractions with screw are stand out because they reach high performances with low liquid-solid ratios, extractions with ultrasound improve the extraction performances and allow obtaining better performances at lower temperatures. Mainly, the use of continuous screw extractors allow efficiencies at industrial level, but still does not offer improvements in terms of selectivity regarding the use of extractors type batch. This problem can be solved by incorporating an acoustic system; however, the low liquid-solid ratios of a continuous extraction system are not favorable for the application of the acoustic field. The conditions in which is profitable to use ultrasound are not offered by countercurrent extraction systems available at present. This shows that it is convenient to introduce some connection that allows the inclusion of both extraction techniques to obtain efficiencies in the processes.

The extraction system developed in the present invention efficiently integrates the advantages of a continuous system (low global liquid-solid ratio) with the advantages of a batch system (specific areas of high liquid-solid ratio). This means, advantage is taken while the global ratio is low 1:5 there are flood areas where the ratio reaches a value near 1:15. this make feasible the application of a cavitative acoustic field in the flooded area, overcoming this way the disadvantages of extended areas and volumes for the propagation of the acoustic wave, factors that make prohibitive the use of this technique because of investment requirements.

This new way of operation not disclosed in the previous art, constitutes one of the most relevant aspects of the present invention. By join the both extraction techniques together, it is possible to use the areas of liquid accumulation of the system to apply, this way, efficiently, the acoustic field in a reduced section of the apparatus. This optimizes the extraction in terms of both power and effectiveness of the investment, producing a new feasible alternative for the industrial establishment of the technology. The potential of the invention reduces the necessity of thermal power, keeps the global liquid-solid ratio, minimizes the area of acoustic propagation, its power and consequently the magnitude of the investment required in the installation and operation phases. Additionally, the combined use of both extraction techniques allows obtaining fewer amounts of non wanted components, improving the purity of the active component in the extracted solids.

The field of application of the present invention is quite wide, since it considers numerous industries performing extractions in the agribusiness.

A more detailed explanation of the invention is provided in the following detailed descriptions and appended claims taken in conjunction with the accompanying drawings.

### BRIEF DESCRIPTION OF THE DRAWINGS

The drawings attached are included to give a better comprehension of the invention, being incorporated as part of this invention and its implementations, and together with the description explain its principles.

FIG. 1 corresponds to a diagram of the cell structure of a vegetal natural product of the previous art treated with a countercurrent extraction method where the extraction means is water.

FIG. 2 correspond to a diagram of the vegetal cell structure of a product of a previous art treated with ultrasounds where the extraction means is water.

FIG. 3 is a drawing of the side view of the bottom part of the equipment, without driving elements for better quality purposes.

FIG. 4a is a drawing of a side view of the bottom part of the equipment without driving elements, and FIG. 4b is a drawing of the elevation view of the bottom part of the equipment without driving elements, where it is illustrated the possibility of radiating from the sides of the extractor.

FIG. 6, corresponds to a preliminary study to establish the influence of the ultrasounds in the extraction process of saponins and solids removable from the Soapbark Tree. The figure compares the traditional method corresponding to 3 [hrs] of extraction at 60° C. and the ultrasonic method corresponding to 20 [min] of extraction at room temperature. In the graphic it is also presented the outcomes of the extraction at room temperature (20° C.) without ultrasounds.

FIG. 7, corresponds to a study to establish the influence of ultrasounds in the process of saponin extraction from the Soapbark tree having as a variable, the concentration of the sample. (Results with a 95% of confidence). Sampling is performed at room temperature.

FIG. 8, corresponds to a study to establish the influence of ultrasounds in the purity of extraction from the Soapbark tree. The figure establishes the relation between purity and granulometry. (Results with a 95% confidence). Sampling is performed at room temperature.

FIG. 9 correspond to a study of the influence of ultrasounds in the saponin extract by HPLC method.

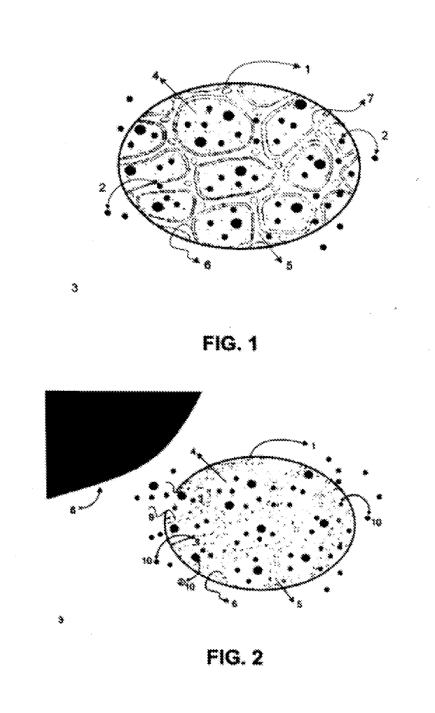

### DETAILED DESCRIPTION OF THE INVENTION

The following is a detailed description and explanation of the preferred embodiments of the invention and best modes for practicing the invention.

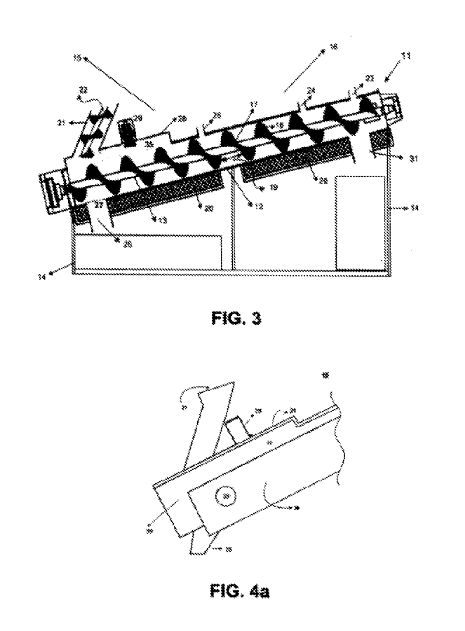

The apparatus of this present invention is shown in FIG. 3. The apparatus (11) consist of a continuous extractor on countercurrent assisted by an acoustic system. It is important to mention that units of screw extractors commercially available at present can be modified and work with beneficial results incorporating to the apparatus an acoustic transducer system with the modifications described as follows.

The apparatus (11), is provided with a case (12) with a groove-cratered shape or cylindrical which contains and supports a first helical conveyor screw (13). The case (12) of the apparatus (11) can be any structure able to contain the first helical conveyor screw (13) which can be bended at different angles thanks to the support structure (14) with adjustable bending means. The bending angle of the screw will depend exclusively on the characteristics of the raw material to be extracted, being that angle between the range of 5° to 85°, the idea is to have the rest of the apparatus be partially submerged with the solvent, thus, an infinitesimal volume of the material to be extracted is always with a volume of solvent without saturating, avoiding the extraction process to be locally suspended. In a preferable mode, the angle is 15°.

The material that goes into the apparatus (11) is raw material made of any natural source, in which some type of product can be extracted, may this be of a vegetal, fruit or organic nature, for example: coffee, tea, tobacco, almonds, fruit, wood, plants or parts of them, or a combination thereof.

In order to move the material along the apparatus (11), an engine provides a rotatory movement to the first helical conveyor screw (13). The engine can be hydraulic, electrical or any other device able to provide a unidirectional rotatory movement to the first helical conveyor screw (13), in such way that allows moving the raw material on countercurrent with the fluid from the bottom end (15) to the top end (16) of the apparatus (11). The first helical conveyor screw (13) includes a plurality of vanes (17) which all together form such helicoids. Each vane has slots (18) to increase the material-solvent contact. In the preferred application of this invention, the slots have a radial configuration extending perpendicularly to the rotation axis of the screw. This avoids the possibility of product stagnation in them. The distance between vanes (17) depends on the properties of the material to be extracted.

The temperature control means (20) is located around the case (12) of the apparatus (11) and it is designed to heat or cold indirectly the bottom side of it. The means of heat energy transfer can be vapor, water or other similar fluid.

The apparatus (11) has at least one inlet of material hopper (21) which is located over the bottom end (15), in a bended position with an angle δ with respect to the surface thereof. This device allows a continuous entry of the raw material through a second helical conveyor screw (22) similar to the first helical conveyor screw (13) of the apparatus (11). The raw material once extracted is discharged through at least one outlet of material hopper (31) located at the top end (16) of the apparatus (11).

At the top end (16) of the apparatus (11) is located at least a first load line (23) for loading the fresh extraction means. To improve the diffusion of the extracting process of this loading of solving device, this can be located in other positions of the apparatus such as a second load line (24) and/or a third load line (25), which are located in other positions at the top of the case, between the first load means (23) and the superficial portion (28).

At the bottom end (15) of the apparatus (11) there is at least one unload line (26) of the liquid extract, this outlet is provided with a sieve (27) which filtrate the final extract before being evacuated.

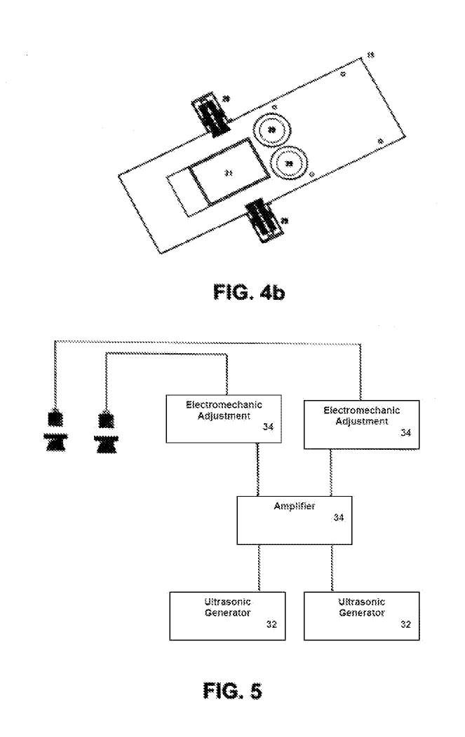

At the bottom end (15) of the apparatus (11) there is a superficial portion (28) of the case (12), over which an acoustic transduction system is located (29), which is benefited by the flood of this little portion of the apparatus favoring the countercurrent extraction. This design allows that all the chips face the acoustic field in the most homogeneous way possible. FIG. 4 illustrates the possibility of radiating from the sides (30) of the extractor's case to increase the radiation intensity.

The transduction system (29) is produced through some high-intensity acoustic transducers. The transducers must radiate the surface of the raw material directly in the extract to avoid that the raw material screened the ultrasonic radiation disfavoring the cavitation and, therefore, the extraction process. The superficial portion (28) that the transduction system supports (29) has a difference in level with the apparatus structure, and between this and the case (12) of the apparatus (11) a mesh is provided (35) to retain the raw material.

In one of the modalities of the present invention, it can be carried out as it is illustrated in FIG. 5, where it is presented a means of excitation for a piezoelectrical transducer. The excitation means consist of a self latching ultrasonic generator (32) for each transducer, a signal amplifier (33) and an electro mechanical matching network (34). Ultrasounds modern systems include an automatic frequency scanning system to ensure that the maximum energy is transmitted to the system.

The acoustic transducer system is provided with an acoustic generation device and an impedance adaption box to facilitate the emission-reception of the acoustic waves used. Such acoustic transducers can have a radiant face in the shape of a disc, stepped horn, plaque. The acoustic transducers have a resonance frequency in a range between 15 and 45 kHz. Acoustic transducer can be located in any section of the apparatus as long as the acoustic cavitation in the extract is favored.

The method of the present invention consists of a continuous extraction on countercurrent assisted by an acoustic system. The natural source to be extracted can be of a vegetal, fruit or organic nature, for example: coffee, tea, tobacco, almonds, fruit, wood, plants or parts of them.

The method has several stages (stages) which are listed below:

### 1. Preparation of the Raw Material

To increase the efficiency of the process, first the natural source must be milled or reduced in size (between 6 and 80 [mm]) By decreasing the size, the contact surface of material-solvent increases, favoring the mass transference of the soluble components from the material to the solvent. Afterwards, and depending on the properties of the biological material to be extracted, the process may or may not include a reduction stage of the material humidity degree through the drying means, either by temperature, pressure, vacuum drying, or any other method that does not alter the properties of the material.

### 2. Adjusting of the Apparatus

In the present invention, the extraction is made in a continuous extractor as the one illustrated in FIG. 3, as previously indicated, the continuous extractor is provided with acoustic capacities. The preferred parameters, and typical for the optimal functioning of the apparatus for extracting active principles, must be established through trials type batch. Each factor must be evaluated according to coherent levels with the industrial process.

The most important factors in the ultrasonic extraction are: granulometry of the product, exposure time to the acoustic field, acoustic power, concentration (solid/liquid) and temperature. In general, each of the variables will depend on the solvent-material-acoustic field interaction, interaction that must be studied in detail. It is important that the material face the acoustic field in the most homogeneous way possible. This stage makes that the ultrasounds affect the cell wall of the material in a way that the following stage of extraction is optimized improving the mass transference.

For the extraction process on countercurrent, the additional factors that must be considered by the time of making the planning of the continuous extraction process are: backmixing, raw material feed rate, bending angle of the screw, insoluble liquid/solid relation, solvent feed rate, product feed rate, screw spin speed and temperature.

In general, to establish efficiency parameters in the extraction process is on countercurrent, it is necessary to study each of the factors and their possible interactions.

### 3. Extraction Process

The process of extraction is performed on countercurrent in a device in which a fluid is poured through one of the ends and through the other, the source material. The raw material goes into the apparatus (FIG. 3) through a hopper (21) located at the bottom part of it, in order to get in contact on countercurrent with the extraction liquid that goes in from the top end of the first load line (23). The extraction liquid preferably used is water, but it can be any other type of solvent according to the process needs, for example: alcohol, chloroform, methanol, acetone, hydro alcohol mixtures.

The bottom end (15) of the case (12) of the apparatus (11) is flooded up to the surface (28) that contains the transducer system (29) leaving its radiant face submerged. The solution formed between the raw material and the extract, allows the conditions for the application of the acoustic field. The acoustic field affects the cell wall increasing the permeability of the material's tissues improving the mass transference and optimizing the countercurrent diffusion. The exposure time of the raw material to the acoustic field and the intensity of it depends exclusively on the properties of the material-liquid mixture in use. Once the source material is subjected to the acoustic field, this continues being elevated on countercurrent with the extraction liquid in such way that the component that delivers (product, solute, raw material, etc.) is in different directions, with the solvent that gathers. The rest of the apparatus must be partially submerged according to the bending angle (70% of the apparatus), thus, an infinitesimal volume of material to be extracted is always with a volume of solvent without saturating, avoiding that the extraction process is locally suspended. During the process, the temperature of the apparatus, through the case, and the solvent can have a range between 16° C. and 80° C. Being a preferred modality, a temperature of 40° C. Once the countercurrent extraction process of the extract is finished, rich in active principles, it pass through the unload line (26) of the apparatus through a filter (27) installed at the end of the extractor and is collected to pass through different purification processes. The exhausted residual material is unloaded through at leas tan unload hopper (31) located at the top end of the extractor.

### APPLICATION EXAMPLE

### Extraction of Active Principles from the Chilean Soapbark Tree Experiments with Ultrasounds Type Batch

The soapbark tree is an endemic tree from Chile (Quillaja Saponaria Molina), whose bark has an important content of saponins. The saponins present in the Soapbark Tree are alkaloid of the triterpenoid type produced during the secondary metabolism of these trees. Saponins have a triterpenic nucleus, with 2 sugar chains joined to this nucleus. Sugar chains give saponins a hydrophilic nature, and the triterpenic nucleus gives a hydrophobic nature, which transform them into an amphoteric molecule. The main effect of these are: reduction of the superficial tension, persisting foaming formation, emulsion of fats and oils, ammonia reduction, activation of the microbial growth, etc.

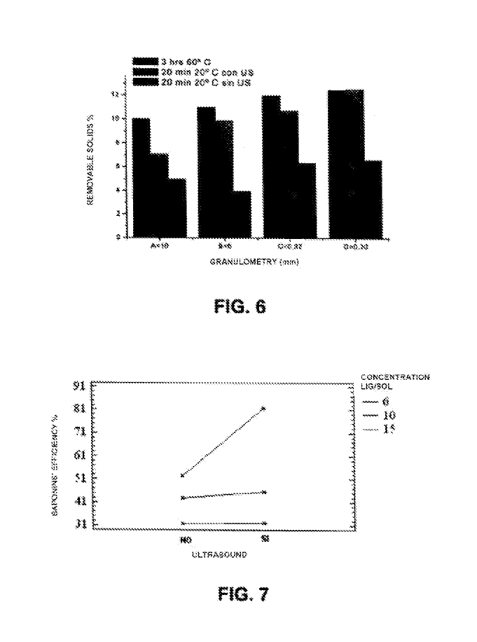

FIG. 6 illustrates the advantages, with respect to the traditional method, that the ultrasound offers to the extraction of active principles from the Soapbark Tree in samples type batch. The results show that when subjecting the chips to an acoustic field for 20 [min] at 20° [C.] they reach total extraction in the case of granulometries type dust, value that indicates the potentiality of the acoustic system respect to the traditional process: 3 [hrs] at 60° [C.]. This demonstrates that there is a clear and significant influence of the ultrasounds in the process of extracting saponins from the Soapbark Tree.

To establish ultrasonic extraction parameters of active principles from the Chilean Soapbark Tree, the factors that influence the process are studied. For this, the extraction process in the plant is simulated by an experimental design. The experimental design is performed with coherent levels that provide effects reproducible at industrial level. The transducer system used for the samples type batch is a vibrator comprised of a piezoelectrical transducer type Langevin coupled to a graded mechanical amplifier which resonance frequency is 20 [kHz]. The influence of each effect and their interactions are studied through experimental factorial designs which allow finding statistical significance in the answers. Each factor must be evaluated according to the coherent levels with the industrial process. The studied factors are: concentration, acoustic power, exposure time to the field and granulometry of the chips.

FIG. 7 illustrates the influence of the ultrasounds in the experimental process developed in our samples type batch according to the concentration of the samples. These results show the feasibility of using the specific areas of high liquid-solid ratio 1:15 for the acoustic treatment of the chips. Being the acoustic cavitation phenomenon of the ultrasonic radiation essential in the extraction process.

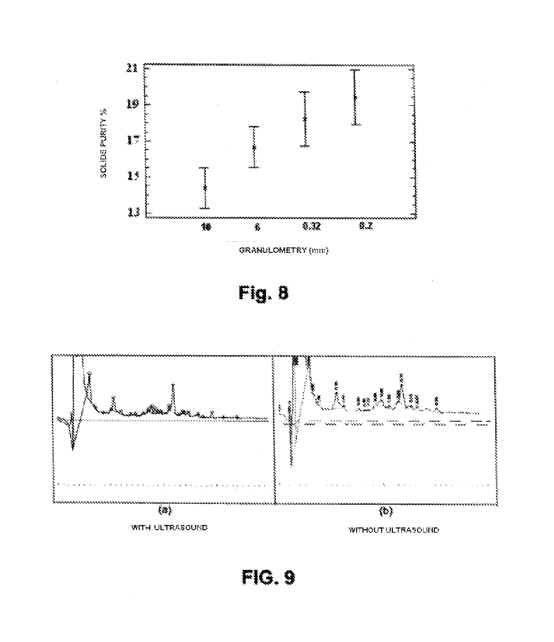

Another important advantage of the ultrasonic method is the selectivity of the samples because there is a decrease in the non desirable components extraction. This is illustrated in FIG. 8 where the purity percentage of the solid extracted increases by decreasing the size of the chip.

Another important issue to consider in the ultrasonic extraction process is the saponin profiles care. FIG. 9 illustrates the saponin profiles of two samples analyzed by HPLC where: a) indicates the sample subjected to an acoustic field and sample b) indicates the profile in standard conditions.

The use of acoustic radiation in the extraction of saponins from the Soapbark Tree has improved the process optimizing it in terms of power as well as effectiveness and investment, producing a feasible alternative for the industrial establishment of the technology. On the other hand, the necessary time for the total extraction of Saponins from the wood has decreased compared to the traditional method of samples type batch (FIG. 6), and the extraction temperature has also been decreased, which optimizes selectivity (FIG. 8), performance and power consumption.

### Procedure of Continuous Extraction on Countercurrent Assisted by Ultrasounds

First of all the wood must be transformed into chips (in this case the bark) to decrease the size of the raw material. Particularly, it is used fibrous chips with diameters in a range between 6 and 80 mm. right after, the product is sieved, in order to eliminate the dust to avoid agglomeration problems in the extract output filter (28).

The extraction begins adding 3 [Kg.] of bark into the apparatus as the solvent is being poured (distilled water) on countercurrent with the raw material per load line (23). The inflow of solvent must be increased until the radiant face of the transducers is completely submerged, the filling level of the pool must be kept constant to avoid variations in the acoustic impedance of the means, taking into account the possibility of adding more solvent to different flows in different parts of the apparatus (24-25) to improve this way the water-chips contact. The water temperature must be of 40° C. to avoid the formation of microorganisms in the final extract. It is important to work at high L/IS (liquid/insoluble solids) concentrations because the wood is capable of absorbing great amount of liquid increasing in size and decreasing the possibilities that all barks coming up through the screw face the solvent, preventing thereby the mass transfer phenomenon.

With the raw material and the arranged Systems as described, the acoustic field is Reddy to be applied. The inflow of the solvent and the outlet of the extract must be kept constant, this way it is avoided that the extractor changed its filling volume. As the raw material goes through it finds the solvent purer each time, facilitating the extraction. The final extract, rich in active principles, pass through a filter (27) installed at the end of the extractor and is collected to be pasteurized later. The exhausted wood is unloaded through the unload hopper (31) located at the top end of the apparatus.

The extractor, with an acoustic system coupled, allows performing extractions with high performances, low temperatures, it uses the liquid accumulation of the system to apply efficiently the acoustic fields in a reduced section of the apparatus, optimizing its application in terms of potency as well as effectiveness and investment, producing a feasible alternative for the industrial scalability of the technology.

Although embodiments of the invention have been shown and described, it is to be understood that various modifications, substitutions, and rearrangements of parts, components, and/or process (method) steps, as well as other uses, shapes, construction, and design of the method and apparatus for extracting active principals from natural sources can be made by those skilled in the art without departing from the novel spirit and scope of this invention.

1. An apparatus for the extraction of active principles from natural sources using an extractor on countercurrent assisted by an acoustic transducer system, which allows the application of an cavitative acoustic field in the area flooded with the material comprised of the raw material of the natural product and a solvent extraction means, where such apparatus is CHARACTERIZED in that it comprises:
a case that surround and supports a first helical conveyor screw with a plurality of vanes which form such helicoids, being such case bended in an angle between 5° and 85°, having the case a bottom end and a top end;
at least an inlet hopper of the material is available over the bottom end of the apparatus in a bended way respect to the top surface of such apparatus, having the inlet hopper a second helical conveyor screw;
at least an outlet hopper to unload the processed material, which is located at the top end of the case;
at least one first load line to load such solvent extraction means located at the top end of the case;
at least one unload line of the liquid extract, located at the bottom end, being that unload line provided with a sieve that filtrates the liquid extract; and
an acoustic transducer system to produce cavitation located at the bottom end over a superficial portion of the case.

2. An apparatus for the extraction of active principles, according to claim 1, CHARACTERIZED in that such apparatus is supported by a supporting structure, with adjustable bending means.

3. An apparatus for the extraction of active principles, according to claim 1, CHARACTERIZED in that such transducer system, comprises high intensity acoustic transducers.

4. An apparatus for the extraction of active principles, according to claim 1, CHARACTERIZED in that such superficial portion that supports the transducer system has a difference in level with the apparatus structure.

5. An apparatus for the extraction of active principles, according to claim 4, CHARACTERIZED in that between such superficial portion and the case a mesh is available that retains the raw material.

6. An apparatus for the extraction of active principles, according to claim 1, CHARACTERIZED in that the acoustic transducer system has excitation means in a self latching ultrasonic generator for the transducers, a signal amplifier and an electromechanical adaptation network.

7. An apparatus for the extraction of active principles, according to claim 1, CHARACTERIZED in that it has an engine that inject a rotatory movement to the first helical conveyor screw.

8. An apparatus for the extraction of active principles, according to claim 1, CHARACTERIZED in that each vane of such plurality of vanes, has slots to increase the contact between the raw material and the solvent extraction means.

9. An apparatus for the extraction of active principles, according to claim 8, CHARACTERIZED in that such slots have a radial configuration extending perpendicularly to the rotation axis of the first helical screw for help avoiding product stagnation in such slots of such vanes.

10. An apparatus for the extraction of active principles, according to claim 1, CHARACTERIZED in that it also comprises temperature control means, located around the case.

11. An apparatus for the extraction of active principles, according to claim 10, CHARACTERIZED in that the heat energy transference means is vapor, water or other similar fluid.

12. An apparatus for the extraction of active principles, according to claim 1, CHARACTERIZED in that the apparatus also comprises a second load line and/or a third load line for the solvent extraction load means, which are located in other positions of the surface of the case, between the first means and the load means and the top superficial portion.

13. An apparatus for the extraction of active principles, according to claim 1, CHARACTERIZED in that said acoustic transducer system emits an acoustic field and the acoustic field is irradiated from the top superficial portion and from the sides of the case.

14. An apparatus for the extraction of active principles, according to claim 3, CHARACTERIZED in that such acoustic transducers may have a radiant face in the shape of a disc, stepped horn, plaque or similar.

15. An apparatus for the extraction of active principles, according to claim 3, CHARACTERIZED in that such acoustic transducers have a resonance frequency in a range between 15 and 45 kHz.

16. An apparatus for the extraction of active principles, according to claim 3, CHARACTERIZED in that such acoustic transducers are located in any section of the apparatus as long as it favors the acoustic cavitation in the extract.

17. An apparatus for the extraction of active principles, according to claim 1, CHARACTERIZED in that the case is bended to an angle of 15°.

18. A method for extracting active principles from natural sources using an extractor on countercurrent assisted by an acoustic transducer system, in an apparatus that allows the application of an cavitative acoustic field in the flooded area with the material comprised of raw material of the natural product and a solvent extraction means, where such apparatus has at least one inlet of such material, at least one inlet of such solvent extraction means, an outlet of processed residual material and at least one outlet of liquid extract, CHARACTERIZED in that it comprises the following steps:
(a) downsizing such material comprised of the raw material of the natural product;
(b) loading such solvent extraction means for at least one inlet of such solvent extraction means in a top end of the apparatus, and loading such material for at least one inlet of such material in a bottom end of the apparatus;
(c) flooding with such solvent extraction means and such material until the top portion of the surface located at the bottom end of the apparatus, that such acoustic transducer system until its radiant face is submerged;
(d) applying an acoustic field to the solution formed between the solvent extraction means and the material;
(e) continuing the extraction on countercurrent between at least one inlet of the solvent extraction means of the apparatus and at least one outlet of the processed residual material.
(f) collecting the extracted liquid obtained; and
(g) collecting the exhausted residual material.

19. A method according to claim 18, CHARACTERIZED in that to establish the application conditions of the acoustic field, the bottom end of the equipment is flooded with the extraction liquid and the material.

20. A method according to claim 18, CHARACTERIZED in that such flood level is kept constant kept during the process.

21. A method according to claim 18, CHARACTERIZED in that the exposure time of the raw material to the acoustic field is not higher than the time of extraction on countercurrent.

22. A method according to claim 18, CHARACTERIZED in that the method includes a prior step of defining at least the parameters of: granulometry of the product, exposure time to the acoustic field, acoustic power, concentration (solid/liquid) and temperature.

23. A method according to claim 24, CHARACTERIZED in that the method also includes the definition of at least the parameters: feed rate of the material, bending angle of the apparatus, liquid/insoluble solid relations, feed rate of the solvent extraction means and screw spin speed.

24. A method according to claim 18, CHARACTERIZED in that the method also includes a partial flooding of the rest of the apparatus according to the bending angle.

25. A method according to claim 24 CHARACTERIZED in that such angle is selected between the range of 5° to 85°.

26. A method according to claim 24, CHARACTERIZED in that such angle is 15°.

27. A method according to claim 18, CHARACTERIZED in that once the extraction on countercurrent is finished, the liquid extract obtained, rich in active principles, and thereafter passing the liquid extract through a unload line of the apparatus through a filter installed at the bottom end of the apparatus.

28. A method according to claim 18, CHARACTERIZED in that the exhausted residual material is unloaded through an unload hopper located at the top end of the apparatus.

29. A method according to claim 18, CHARACTERIZED in that the stage (a) also includes reducing the humidity degree of such material through drying means.

30. A method according to claim 18, CHARACTERIZED in that the extraction liquid used is water.

31. A method according to claim 18, CHARACTERIZED in that the extraction liquid used is selected from the group consisting of: alcohol, chloroform, methanol, acetone, hydro alcoholic mixtures and combinations thereof.

32. A method according to claim 18, CHARACTERIZED in that the solid/liquid relation in the acoustic transducer part must favor the acoustic cavitation.

33. A method according to claim 18, CHARACTERIZED in that the material includes a range of size between 6 mm and 80-mm.

34. A method according to claim 18, CHARACTERIZED in that in step (d) the acoustic waves influence frontally the surface of the product.

35. A method according to claim 18, CHARACTERIZED in that the method includes a process temperature between approximately 16° C. and 80° C.
